(19) United States
(12) Patent Application Publication
(10) Pub. No.: US 2025/0221631 A1
(43) Pub. Date: Jul. 10, 2025

(54) HEART RATE SENSING SYSTEM AND METHOD

(71) Applicants: HYUNDAI MOTOR COMPANY, Seoul (KR); KIA CORPORATION, Seoul (KR); SL Corporation, Daegu (KR)

(72) Inventors: Sung Ho Park, Seoul (KR); Jung Kwon Son, Seoul (KR); Jong Pil Kim, Seoul (KR); Heung Yong Kim, Daegu (KR); Myong Guan Jeong, Busan (KR)

(73) Assignees: HYUNDAI MOTOR COMPANY, Seoul (KR); KIA CORPORATION, Seoul (KR); SL Corporation, Daegu (KR)

(21) Appl. No.: 18/804,986

(22) Filed: Aug. 14, 2024

(30) Foreign Application Priority Data
Jan. 5, 2024 (KR) 10-2024-0002140

(51) Int. Cl. A61B 5/024; A61B 5/00

(52) U.S. Cl. CPC A61B 5/02433; A61B 5/7203; A61B 5/7267; A61B 2503/22

(57) ABSTRACT

Biometric information is obtained through a plurality of near-infrared ray irradiations, so that a vehicle occupant's biometric information can be stably acquired even in various lighting environments such as daytime, night, sunset, sunrise, shade, and when illuminated by street lights. In addition, through the plurality of near-infrared ray irradiations, the biometric information according to a hemoglobin absorption rate at different skin penetration depths is acquired, and the occupant's heart rate is determined based on multiple pieces of biometric information, so that a system and method for sensing a heart rate with minimized noise and improved accuracy are disclosed.

### CROSS REFERENCE TO RELATED APPLICATION

This application claims priority to Korean Patent Application No. 10-2024-0002140, filed Jan. 5, 2024, the entire contents of which are incorporated herein for all purposes by this reference.

### BACKGROUND

### Technical Field

The disclosure relates to a system and method for sensing the heart rate of an occupant.

### Description of the Related Art

Typically, a user wears a wearable device on his or her body to sense heart rate and monitor health indicators. In particular, in the case of a heart rate sensor, the user's heart rate can be measured and the disease prognosis according to the user's heart rate can be observed in advance.

In particular, if the driver experiences a cardiac event such as cardiac arrest due to an abnormal heart rate while driving the vehicle, a secondary accident may occur, resulting in a great risk of harm to the driver and others.

For this purpose, a device to monitor the driver's condition is applied to or included in the vehicle. The monitoring device uses a camera to photograph the driver's face, condition, and posture, and analyzes them to check the driver's condition.

However, there are limitations in checking the driver's condition using only the images captured through cameras. Specifically, when using a camera, it is impossible to directly check the heart rate because it only recognizes the driver's face, condition, and posture.

Meanwhile, there are methods of using light to measure the driver's heart rate, which are divided into contact methods and a non-contact methods.

In the case of the contact method, there is a problem that driving inconvenience occurs because the driver must wear a wearable device in the vehicle.

The non-contact method measures the driver's heart rate by irradiating light of a specific wavelength to the driver's skin and sensing the amount of light transmitted and reflected.

However, when using an RGB camera sensor to monitor occupants inside a vehicle according to the non-contact method, errors in heart rate sensing may occur due to insufficient or saturated light depending on the surrounding environment.

Further, when using a near-infrared camera sensor, information for heart rate sensing is relatively insufficient, resulting in lower accuracy.

The information disclosed in this Background of the disclosure section is provided only to enhance understanding of the general background of the disclosure. Thus, the information disclosed in this Background of the disclosure section should not be understood as an acknowledgement that information disclosed therein forms the prior art already known to a person having ordinary skill in the art.

### SUMMARY

An object of the present disclosure is to provide a system and method for sensing a heart rate that can stably obtain an occupant's biometric information and accurately determine the occupant's heart rate even in various lighting environments.

In order to achieve the above object, a system for sensing a heart rate according to the disclosure may include a sensing target region-determination unit that configured to check a position of a subject and set a sensing target region. The system further includes: an optical information collection unit configured to acquire optical information in each of multiple infrared wavelength bands over time using infrared rays in the different wavelength bands in the sensing target region; and a derivation unit configured to classify a plurality of pieces of optical information by frequency and filters a noise signal to derive heart rate information.

The sensing target region-determination unit may be configured to check a face position of the subject and may set the sensing target region based on a feature point derived by applying the face of the subject to a trained machine learning algorithm model.

The sensing target region-determination unit may be configured to derive the sensing target region based on the machine learning algorithm model trained according to a distribution of blood vessels in the face.

The optical information collection unit may include a plurality of lighting devices configured to irradiate the infrared rays in the different wavelength bands, and a detection unit configured to sense reflected light in which the infrared rays are reflected from the subject as the optical information.

The plurality of lighting devices may be configured to irradiate near-infrared rays in different wavelength bands, respectively.

The plurality of lighting devices may be set to irradiate the near-infrared rays in different wavelengths in a range of greater than 760 nm and less than 2,500 nm, wherein a first one of the wavelengths is configured to penetrate relatively shallow and a second one of the wavelengths is configured to penetrate a relatively deep depth so that the subject is divided into shallow depth and deep depth.

The plurality of lighting devices may be set to exclude a visible light wavelength range from a solar spectrum.

The optical information collection unit may be operated using a combination of a plurality of lightings over time, with the plurality of lighting devices being alternatingly turned on and off or simultaneously turned on.

The optical information collection unit may exclude a control in which the respective lighting devices are turned off simultaneously from the plurality of lighting combinations, and the plurality of lightings may be combined so that a frequency of turning on and off each lighting device is minimized.

The derivation unit may be configured to receive a brightness value for each lighting combination according to the optical information obtained for each lighting combination from the optical information collection unit, and may derive the heart rate information through frequency classification and noise signal filtering processes.

In another embodiment of the present disclosure, a method for sensing a heart rate may include: a sensing target region-determination step of checking a face position of a subject and setting a sensing target region on the face of the subject; an optical information collection step of acquiring optical information in each of multiple infrared wavelength bands through a lighting device irradiating infrared rays in different wavelength bands; and a derivation step of classifying a plurality of pieces of optical information by frequency and filtering a noise signal to derive heart rate information.

In the sensing target region-determination step, the sensing target region may be set based on a feature point derived by applying the face of the subject to a trained machine learning algorithm model.

In the optical information collection step, the optical information may be obtained by allowing a plurality of lighting devices to alternatingly turn on and off or turn on simultaneously to operate in a plurality of light combinations over time.

In the optical information collection step, a control in which the respective lighting devices are turned off simultaneously may be excluded from the plurality of lighting combinations, and the plurality of lightings may be combined so that a frequency of turning on and off each lighting device is minimized.

In the derivation step, a brightness value for each lighting combination may be received according to the optical information obtained for each lighting combination, and the heart rate information may be derived through frequency classification and noise signal filtering processes.

The system and method for sensing a heart rate configured as described above acquire biometric information through a plurality of near-infrared ray irradiations, so that the occupant's biometric information can be reliably acquired even in various lighting environments such as daytime, night, sunset, sunrise, shade, and when the occupant is illuminated by street lights.

In addition, through the plurality of near-infrared ray irradiations, the biometric information according to a hemoglobin absorption rate at different skin penetration depths is acquired, and the occupant's heart rate is determined based on multiple pieces of biometric information, thereby minimizing noise and improving accuracy.

### BRIEF DESCRIPTION OF THE DRAWINGS

FIG. 1 is a configuration diagram of a system for sensing a heart rate according to the present disclosure.

FIG. 2 is a diagram illustrating an example of a sensing target region-determination unit and optical information collection unit in the system for sensing a heart rate illustrated in FIG. 1.

FIG. 3 is a diagram illustrating a sensing target region according to the disclosure.

FIG. 4 is a diagram illustrating an example of a solar spectrum.

FIG. 5 is a diagram illustrating a lighting combination of a lighting device according to an embodiment of the present disclosure.

FIG. 6 is a diagram illustrating a lighting combination of a lighting device according to another embodiment of the present disclosure.

FIG. 7 is a diagram illustrating a lighting combination of a lighting device over time according to an embodiment of the present disclosure.

FIG. 8 is a diagram illustrating a classification for each channel in a lighting combination of a lighting device according to an embodiment of the present disclosure.

FIG. 9 is a diagram illustrating a derivation of a brightness value for each channel according to an embodiment of the disclosure.

FIG. 10 is a diagram illustrating removal of a direct current component for each channel according to an embodiment of the disclosure.

FIG. 11 is a diagram illustrating a frequency distribution and frequency analysis and heart rate derivation for each channel according to an embodiment of the disclosure.

FIG. 12 is a diagram illustrating heart rate derivation by collecting the results of each channel according to an embodiment of the disclosure.

FIG. 13 is a flowchart of a method for sensing a heart rate according to the disclosure.

### DETAILED DESCRIPTION

Hereinafter, embodiments of the disclosure are described in detail with reference to the attached drawings. The same or similar components are given the same reference numbers and a redundant description thereof is omitted.

The suffixes “module” and “unit” of elements herein are used for convenience of description and thus can be used interchangeably and do not have any distinguishable meanings or functions. The term “unit” or “module” used in this specification signifies one unit that processes at least one function or operation, and may be realized by hardware, software, or a combination thereof. The operations of the method or the functions described in connection with the forms disclosed herein may be embodied directly in a hardware or a software module executed by a processor, or in a combination thereof.

Further, in the following description, if it has been determined that a detailed description of known techniques associated with the disclosure would unnecessarily obscure the gist of the disclosure, a detailed description thereof has been omitted. In addition, the attached drawings are provided to enhance understanding of embodiments of the disclosure and do not limit technical spirits of the disclosure. Accordingly, the embodiments should be construed as including all modifications, equivalents, and alternatives falling within the spirit and scope of the embodiments.

While terms, such as “first”, “second”, etc., may be used to describe various elements, such elements must not be limited by the above terms. The above terms are used only to distinguish one element from another.

When an element is “coupled” or “connected” to another element, it should be understood that a third element may be present between the two elements although the element may be directly coupled or connected to the other element. When an element is “directly coupled” or “directly connected” to another element, it should be understood that no element is present between the two elements.

The singular forms are intended to include the plural forms as well, unless the context clearly indicates otherwise.

In addition, in the specification, it should be further understood that the terms “comprise” and “include” specify the presence of stated features, integers, steps, operations, elements, components, and/or combinations thereof, but do not preclude the presence or addition of one or more other features, integers, steps, operations, elements, components, and/or combinations. When a component, device, element, or the like of the present disclosure is described as having a purpose or performing an operation, function, or the like, the component, device, or element should be considered herein as being “configured to” meet that purpose or perform that operation or function.

A controller may include a communication device configured to communicate with other controllers or sensors, a memory configured to store operating system, logic commands, input and output information, etc., and at least one processor configured to perform determination, calculation, judgement, etc. necessary to control a function assigned to the controller so as to control the function.

Hereinafter a system and method for sensing a heart rate according to an embodiment of the disclosure is described with reference to the attached drawings.

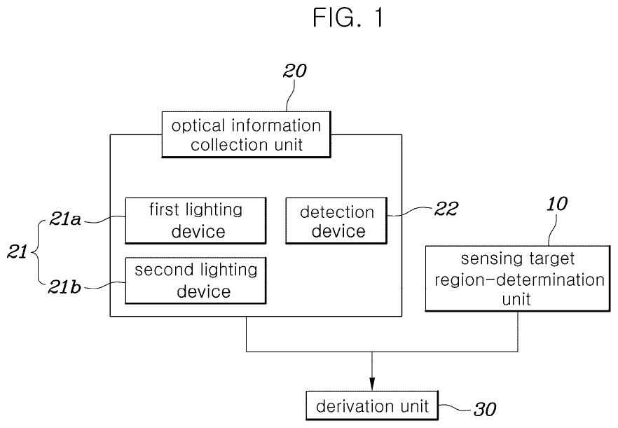

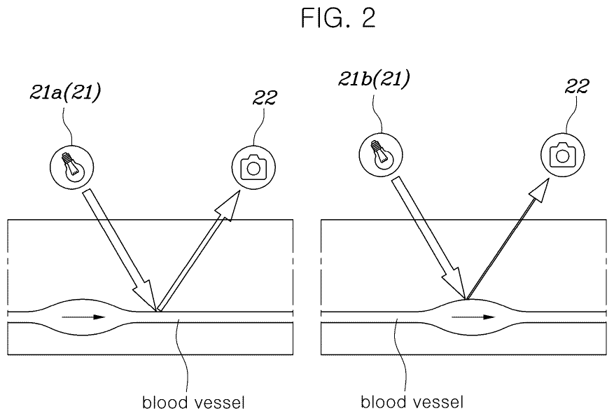

As illustrated in FIGS. 1-3, a system for sensing a heart rate includes a sensing target region-determination unit 10 that checks a position of a subject P and sets a sensing target region S; an optical information collection unit 20 that acquires optical information in each of multiple infrared wavelength bands over time using infrared rays in different wavelength bands in the sensing target region (S); and a derivation unit 30 that classifies a plurality of pieces of optical information by frequency and filters a noise signal to derive heart rate information.

The system for sensing a heart rate includes the sensing target region-determination unit 10, the optical information collection unit 20, and the derivation unit 30. The sensing target region-determination unit 10 and the optical information collection unit 20 collects information to derive the heart rate.

The derivation unit 30 collects or receives the information collected through the sensing target region-determination unit 10 and optical information collection unit 20 to derive heart rate information.

In the disclosure, the subject P may be an occupant in a vehicle.

The sensing target region-determination unit 10 may check the position of the face of the subject P and set the sensing target region S based on the feature points derived by applying the face of the subject P to a trained machine learning algorithm model.

The sensing target region-determination unit 10 may include a camera and may photograph the face of the subject P. The sensing target region-determination unit 10 may be located in front of the subject P inside the vehicle, and may be configured to track the face of the subject P.

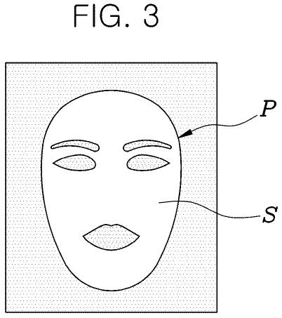

Referring to FIG. 3, the sensing target region-determination unit 10 may check the position of the face of the subject P and sets the sensing target region S by applying the checked face to the trained machine learning algorithm model.

When checking the face position of the subject P, the positions of the two eyes of the subject P may be used as a reference for the feature point. After removing noise including the eyes and eyebrows based on the feature point, the skin of the subject P may be set as the sensing target region S.

Through this, the sensing target region-determination unit 10 may derive the sensing target region S based on a machine learning algorithm model trained according to the distribution of blood vessels in the face.

In other words, the sensing target region S may be the skin where the blood vessels are distributed on the face of the subject P, and only the skin region of the subject P may be estimated based on the trained machine learning algorithm model.

In this way, the sensing target region-determination unit 10 may photograph the face of the subject P, collect facial information of the subject P as learning data based on the feature points, and create the algorithm model through regression analysis or machine learning by using the collected learning data.

In this case, the regression analysis algorithm may include simple linear regression analysis, multiple linear regression analysis, logistic regression analysis, and the like, and the machine learning algorithm may include artificial neural networks, decision trees, genetic algorithms, random forests, deep learning, and the like.

In the disclosure, the sensing target region S according to the subject P is set through the trained machine learning algorithm.

In this way, the sensing target region-determination unit 10 photographs the face of the subject P and sets the sensing target region S according to the subject P through the trained machine learning algorithm model, so that the region where information for heart rate measurement may be collected from the face of the subject P may be limited, and noise may be minimized when collecting heart rate information through the optical information collection unit 20.

The optical information collection unit 20 acquires optical information in each of multiple infrared wavelength bands over time using infrared rays in different wavelength bands in the sensing target region S set by the sensing target region-determination unit 10.

The optical information collection unit 20 may use an infrared sensor or an infrared sensor and an RGB (Red, Green, Blue color) sensor to acquire optical information in which light irradiated on the subject P is reflected. In other words, the optical information collection unit 20 may use a plurality of infrared sensors to acquire the optical information according to the infrared light in different wavelength bands or use an infrared sensor and an RGB sensor to acquire optical information using the infrared light in different wavelength bands and the color of light in three (e.g., red, green, and blue) wavelength bands.

According to the present disclosure, infrared rays in different wavelength bands are utilized, and the infrared rays in different wavelength bands are combined for a certain period over time, and the optical information is obtained by emitting the infrared rays for each combination.

In detail, the optical information collection unit 20 may be configured to include a plurality of lighting devices 21 or lighting means that irradiate infrared rays in different wavelengths, and a detection device 22 that senses reflected light in which the infrared rays are reflected from the subject P as the optical information.

In this way, the optical information collection unit 20 may be configured to include the plurality of lighting devices 21 and the detection device 22. Each lighting device 21 may be configured to irradiate infrared rays in different wavelength bands, and the detection device 22 may be configured to detect the infrared rays emitted from each lighting device 21. Accordingly, multiple detection devices 22 may be provided or included in the heart rate sensing system, and each detection device 22 may be set to detect the infrared rays in different wavelength bands.

The plurality of lighting devices 21 may each be composed of light sources that irradiate near-infrared rays in different wavelength bands.

According to an embodiment, the plurality of lighting devices 21 is configured to include a first lighting device 21a and a second lighting device 21b. The first lighting device 21a and second lighting device 21b may be configured to irradiate the near-infrared rays in different wavelength ranges.

In other words, the first lighting device 21a irradiates the near-infrared rays in a first wavelength band, and the second lighting device 21b irradiates the near-infrared rays in a second wavelength band. The first and second wavelength bands may be determined by excluding the visible light wavelength range from the solar spectrum.

In detail, the plurality of lighting devices 21 may be set to irradiate the near-infrared rays in different wavelengths in the range of greater than 760 nm and less than 2,500 nm, so that the subject P may be divided into shallow depth and deep depth.

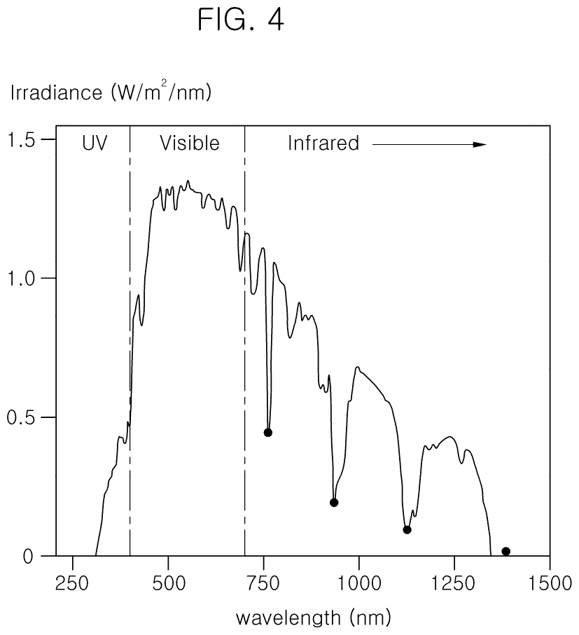

Referring to FIG. 4, in the solar spectrum, the range where the irradiance is below a certain amount may be seen as excluding the visible light wavelength range. For example, the wavelength range may be determined within 760 nm to 2500 mm, including 760 nm, 940 nm, and 1,130 nm. Accordingly, the first wavelength band of the first lighting device 21a and the second wavelength band of the second lighting device 21b may be set to different wavelength bands within the range of 760 nm to 2500 mm, excluding the visible light wavelength range.

For example, the first wavelength band of the first lighting device 21a may be a short wavelength with a shallow depth and may be set to 760 nm. The second wavelength band of the second lighting device 21b may be a long wavelength that has a relatively deep penetration into the skin of the subject P compared to a short wavelength and may be set to 1,130 nm.

Through this, the plurality of lighting devices 21 may sense the heart rate information based on blood flow within the blood vessels positioned in the dermis, subcutaneous tissue, etc. depending on the skin depth of the subject P. Since the skin penetration depths of the near-infrared rays in different wavelength bands are different from each other, the accuracy of heart rate measurement may be improved with the optical information checked through the plurality of lighting devices 21.

Here, the optical information collection unit 20 may be operated using a combination of a plurality of lightings over time, with the plurality of lighting devices 21 being alternatingly turned on/off or simultaneously turned on.

In addition, the optical information collection unit 20 excludes the control in which the respective lighting device 21 are turned off simultaneously from the plurality of lighting combinations. The plurality of lightings may be combined so that the frequency of turning on and off each lighting device 21 is minimized.

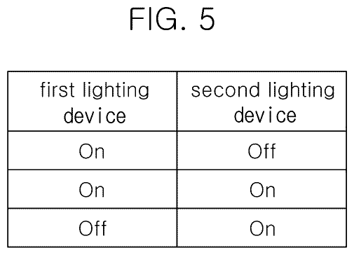

In the disclosure, the lighting device 21 may consist of a plurality of lighting devices. As illustrated in FIG. 5, when the plurality of lighting devices 21 consists of a first lighting device 21a and a second lighting device 21b, there may be three lighting combinations.

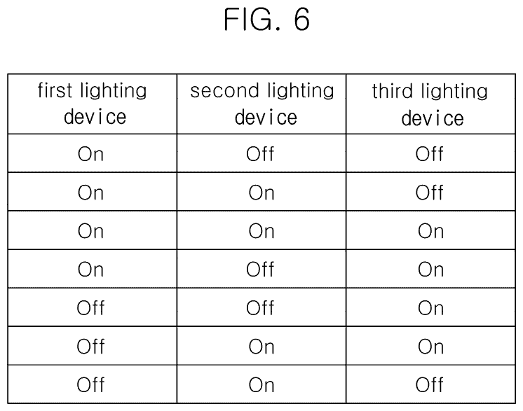

According to another embodiment, if a lighting combination includes three lighting devices 21, as illustrated in FIG. 6, seven lighting combinations may exist. The number of lighting devices 21 may be determined in advance through experimentation, depending on the accuracy of the heart rate determination according to or based on the plurality of pieces of optical information.

The lighting device 21 consists of a plurality of lighting devices, so that the frequency of turning on and off of each lighting devices 21 is minimized to reduce noise generation. In other words, when N lighting devices 21 of different wavelength bands are applied, the lighting device 21 may consist of 2^{N}−1 lighting combinations.

The lighting combination of the optical information collection unit 20 may be repeated at a certain or predetermined period over time. In this case, the period of the lighting combination may be set to at least two times per second based on the minimum target heart rate frequency.

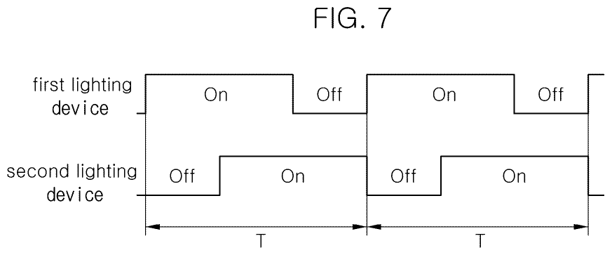

For example, when the lighting device 21 consists of the first lighting device 21a and the second lighting device 21b, the lighting combination of each lighting device 21 is repeated in or over two or more periods of time as illustrated in FIG. 7.

In this way, the lighting device 21 performs classification over time for each channel according to alternating on/off state or simultaneous turning on of the first lighting device 21a and second lighting device 21b.

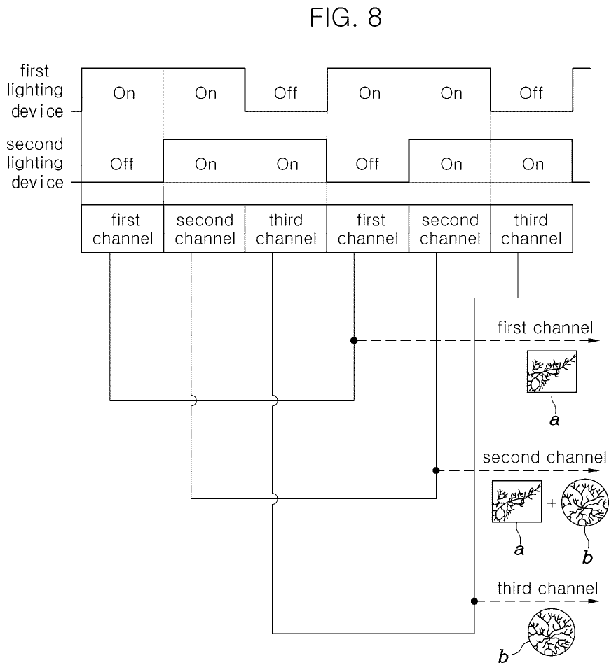

In other words, as illustrated in FIG. 8, in the case of a first channel in which the first lighting device 21a is turned on and the second lighting device 21b is turned off, the first lighting device 21a has a shallow depth of field in the near-infrared wavelength band, so that the biometric information according to an amount of hemoglobin absorbed through the first lighting device 21a may be expressed as ‘a’.

In the case of a second channel in which the first lighting device 21a and second lighting device 21b are turned on simultaneously, the biometric information according to an amount of hemoglobin absorbed through the first lighting device 21a, which has a shallow depth of field in the near-infrared wavelength range, and the second lighting device 21b, which has a deep depth of field in the near-infrared wavelength range, may be expressed as ‘a’ and ‘b’.

In the case of a third channel in which the first lighting device 21a is turned off and the second lighting device 21b is turned on, the second lighting device 21b has a deep depth of field in the near-infrared wavelength band, so that the biometric information according to an amount of hemoglobin absorbed through the second lighting device 21b may be expressed as ‘b’.

Through this, the derivation unit 30 may receive a brightness value for each lighting combination according to the optical information obtained for each lighting combination from the optical information collection unit 20 and may derive heart rate information through frequency classification and noise signal filter processes.

In other words, the derivation unit 30 inputs optical information for a plurality of channels through each lighting device 21 forming the optical information collection unit 20. In this way, the derivation unit 30 receives the brightness value over time for each channel, removes the direct current component of each channel, and removes the noise signal that analyzes the frequency and frequency to obtain heart rate information of the subject P.

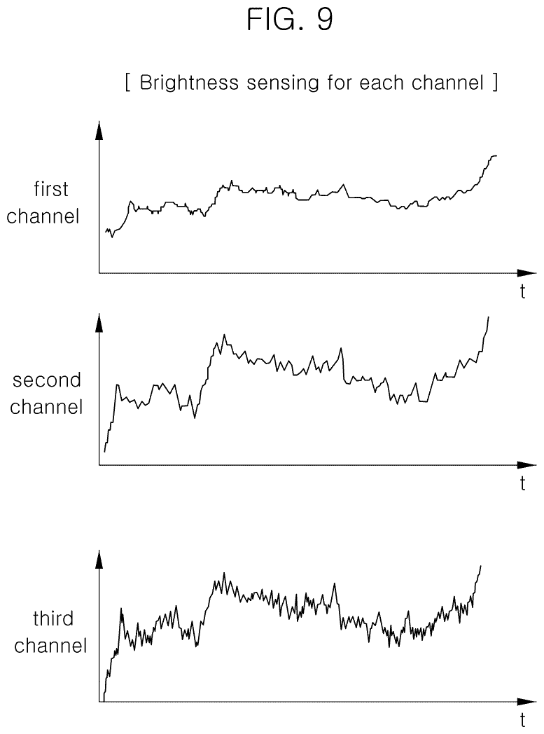

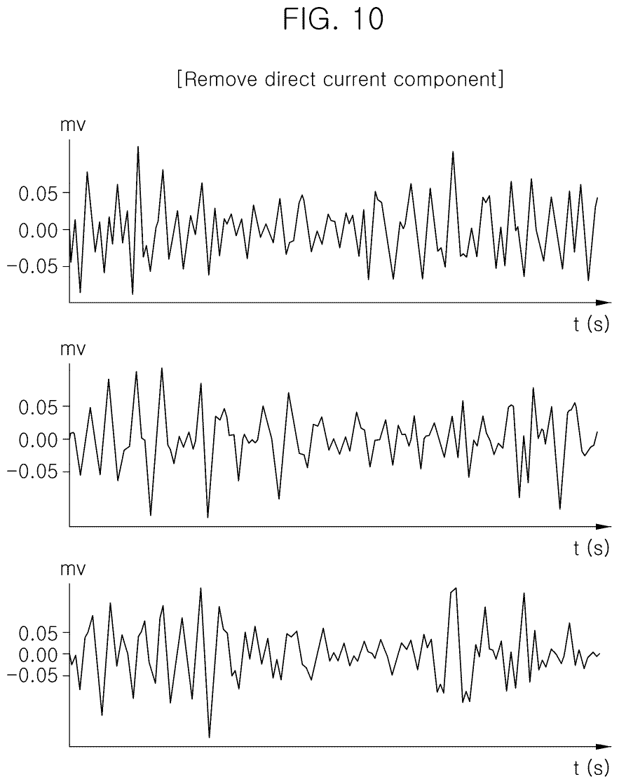

For example, when the optical information collection unit 20 consists of the first lighting device 21a and the second lighting device 21b, if the brightness values for the three channels are derived, respectively, as illustrated in FIG. 9, and a direct current component is removed for each of the three channels to normalize, as illustrated in FIG. 10.

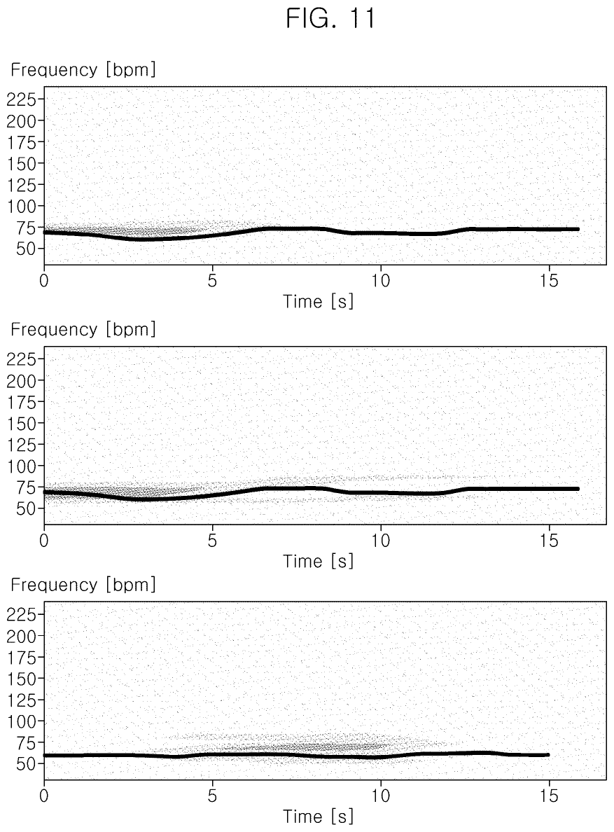

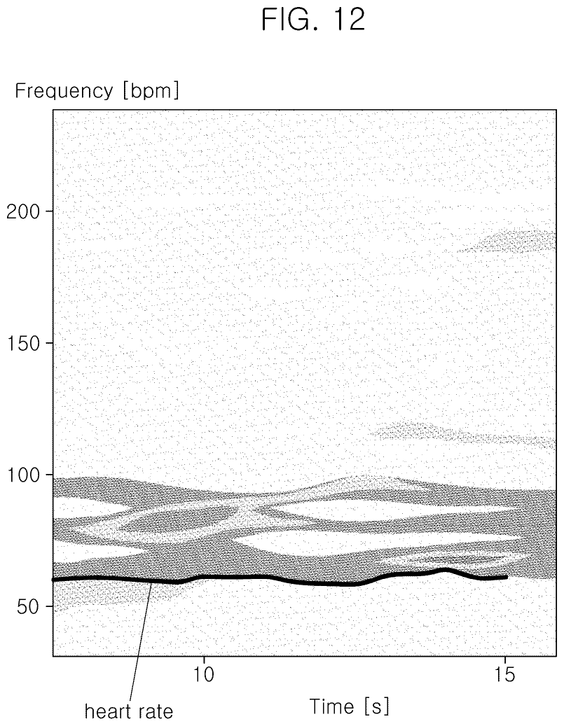

Afterwards, as illustrated in FIG. 11, the frequency distribution and frequency for each channel are analyzed over time, and the respective analyzed results may be collected as illustrated in FIG. 12 to derive the heart rate information of the subject P.

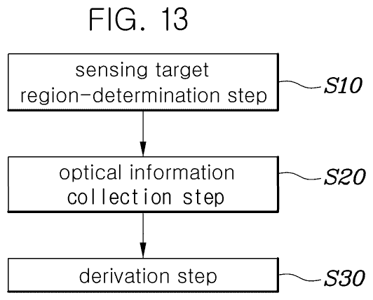

Meanwhile, as illustrated in FIG. 13, a method for sensing a heart rate according to the disclosure may include a sensing target region-determination step (S10) of checking a face position of a subject P and setting a sensing target region S on the face of the subject P; an optical information collection step (S20) of acquiring optical information in each of multiple infrared wavelength bands through the lighting device 21 irradiating infrared rays in different wavelength bands; and a derivation step (S30) of classifying a plurality of pieces of optical information by frequency and filtering a noise signal to derive heart rate information.

Here, in the sensing target region-determination step (S10), the sensing target region S may be set based on a feature point derived by applying the face of the subject P to a trained machine learning algorithm model.

The face of the subject P may be checked through a camera, and the camera may be located in front of the subject P inside the vehicle and may be configured to track the face of the subject P.

In this way, in the optical information collection step (S20), the face position of the subject P is checked, and the checked face is applied to the trained machine learning algorithm model to set the sensing target region S. When checking the face position of the subject P, the positions of the two eyes of the subject P may be used as a reference for the feature point. After removing noise including the eyes and eyebrows based on the feature point, the skin of the subject P may be set as the sensing target region S.

Through this, the sensing target region-determination unit 10 may derive the sensing target region S based on a machine learning algorithm model trained according to the distribution of blood vessels in the face. In other words, the sensing target region S may be the skin where the blood vessels are distributed on the face of the subject P, and only the skin region of the subject P may be estimated based on the trained machine learning algorithm model.

Meanwhile, in the optical information collection step (S20), the optical information may be obtained by allowing the plurality of lighting devices 21 to alternatingly turn on/off or turn on simultaneously to operate in a plurality of light combinations over time.

In addition, in the optical information collection step (S20), a control in which the respective lighting devices 21 are turned off simultaneously is excluded from the plurality of lighting combinations, and the plurality of lightings may be combined so that a frequency of turning on and off each lighting devices 21 is minimized.

In the disclosure, the lighting device 21 may consist of a plurality of light devices. When the plurality of lighting devices 21 consists of the first lighting device 21a and the second lighting device 21b, there may be three lighting combinations.

The lighting combination of each lighting device 21 may be repeated at a certain or predetermined period over time. In this case, the period of the lighting combination may be set to at least two times per second based on the minimum target heart rate frequency.

For example, when the lighting device 21 consists of the first lighting device 21a and the second lighting device 21b, the lighting combination of each lighting device 21 may be repeated in or over two or more periods of time. In this way, the lighting combination according to alternating turning on and off of the first lighting device 21a and the second lighting device 21b or simultaneously turning on the first lighting device 21a and second lighting device 21b may be classified over time for each channel.

Meanwhile, in the derivation step (S30), a brightness value for each lighting combination may be received according to the optical information obtained for each lighting combination, and the heart rate information may be derived through frequency classification and noise signal filtering processes.

In this way, in the derivation step (S30), the brightness value over time is input for each of plurality of channels through each lighting device 21, a direct current component of each channel is removed, and the noise signal for analyzing the frequency part and frequency is removed. Accordingly, the heart rate information of the subject P may be derived.

The system and method for sensing a heart rate configured as described above acquire biometric information through a plurality of near-infrared ray irradiations, so that the occupant's biometric information can be stably acquired even in various (e.g., lighting) environments such as daytime, night, sunset, sunrise, shade, and street lights.

In addition, through the plurality of near-infrared ray irradiations, the biometric information according to a hemoglobin absorption rate at different skin penetration depths is acquired, and the occupant's heart rate is determined based on multiple pieces of biometric information, thereby minimizing noise and improving accuracy.

Although the disclosure has been illustrated and described in connection with several specific embodiments and accompanying drawings, it should be readily understood by those of ordinary skill in the art that various modifications and changes can be made to the embodiments described herein without departing from the spirit and scope of the disclosure defined by the appended claims.

What is claimed is:

1. A system for sensing a heart rate, comprising:
a sensing target region-determination unit configured to check a position of a subject and set a sensing target region;
an optical information collection unit configured to acquire optical information in each of multiple infrared wavelength bands over time using infrared rays in different wavelength bands in the sensing target region; and
a derivation unit configured to classify a plurality of pieces of optical information by frequency and filter a noise signal to derive heart rate information.

2. The system of claim 1, wherein the sensing target region-determination unit is configured to check a face position of the subject and set the sensing target region based on a feature point derived by applying a face of the subject to a trained machine learning algorithm model.

3. The system of claim 2, wherein the sensing target region-determination unit is configured to derive the sensing target region based on the machine learning algorithm model trained according to a distribution of blood vessels in the face.

4. The system of claim 1, wherein the optical information collection unit includes a plurality of lighting devices configured to irradiate the infrared rays in the different wavelength bands, and a detection unit configured to sense reflected light in which the infrared rays are reflected from the subject as the optical information.

5. The system of claim 4, wherein the plurality of lighting devices are configured to irradiate near-infrared rays in different wavelength bands, respectively.

6. The system of claim 5, wherein the plurality of lighting devices are set to irradiate the near-infrared rays in different wavelengths in a range of greater than 760 nm and less than 2,500 nm, wherein a first wavelength of the wavelengths is configured to penetrate a relatively shallow depth and a second wavelength of the wavelengths is configured to penetrate a relatively deep depth so that the subject is divided into shallow depth and deep depth.

7. The system of claim 5, wherein the plurality of lighting devices are set to exclude a visible light wavelength range from a solar spectrum.

8. The system of claim 5, wherein the optical information collection unit is operated using a combination of a plurality of lightings over time, with the plurality of lighting devices being alternatingly turned on and off or simultaneously turned on.

9. The system of claim 8, wherein the optical information collection unit excludes a control in which the respective lighting devices are turned off simultaneously from the plurality of lighting combinations, and the plurality of lightings is combined so that a frequency of turning on and off each lighting device is minimized.

10. The system of claim 5, wherein the derivation unit is configured to receive a brightness value for each lighting combination according to the optical information obtained for each lighting combination from the optical information collection unit and derive the heart rate information through frequency classification and noise signal filtering processes.

11. A method for sensing a heart rate, comprising:
a sensing target region-determination step of checking a face position of a subject and setting a sensing target region on a face of the subject;
an optical information collection step of acquiring optical information in each of multiple infrared wavelength bands through a lighting device irradiating infrared rays in different wavelength bands; and
a derivation step of classifying a plurality of pieces of optical information by frequency and filtering a noise signal to derive heart rate information.

12. The method of claim 11, wherein in the sensing target region-determination step, the sensing target region is set based on a feature point derived by applying the face of the subject to a trained machine learning algorithm model.

13. The method of claim 11, wherein in the optical information collection step, the optical information is obtained by allowing a plurality of lighting devices to alternatingly turn on and off or turn on simultaneously to operate in a plurality of light combinations over time.

14. The method of claim 13, wherein in the optical information collection step, a control in which the respective lighting devices are turned off simultaneously is excluded from the plurality of lighting combinations, and the plurality of lightings is combined so that a frequency of turning on and off each lighting devices is minimized.

15. The method of claim 11, wherein in the derivation step, a brightness value for each lighting combination is received according to the optical information obtained for each lighting combination, and the heart rate information is derived through frequency classification and noise signal filtering processes.
